(19) United States
(12) Patent Application Publication
(10) Pub. No.: US 2026/0226537 A1
(43) Pub. Date: Aug. 6, 2026

(54) CALIBRATION OF A NANOPORE ARRAY DEVICE

(71) Applicant: Oxford Nanopore Technologies PLC, Oxford (GB)

(72) Inventor: Olle Alfred Nordesjö, Oxford (GB)

(73) Assignee: Oxford Nanopore Technologies PLC, Oxford (GB)

(21) Appl. No.: 19/150,554

(22) PCT Filed: Jan. 23, 2024
(86) PCT No.: PCT/GB2024/050173

§ 371 Date: Jul. 24, 2025

(30) Foreign Application Priority Data
Jan. 25, 2023 (GB) 2301095.2

(51) Int. Cl. C12Q 1/6869; G01N 33/487

(52) U.S. Cl. CPC C12Q 1/6869; G01N 33/48721

(57) ABSTRACT

A method of generating a polymer sequence using a nanopore sequencing device, the nanopore array device comprising a nanopore channel, the nanopore channel (35) formed in a membrane (32) separating two ionic solutions (33, 36), the nanopore channel connecting the ionic solutions, the method comprising: translocating a series of polymers through the nanopore channel; generating measurement signals during the translocation of each polymer through the nanopore channel; analysing a set of measurement signals from each polymer taken during the translocation of said polymer through the nanopore channel to determine a scaling factor for each polymer; applying the scaling factor to the measurement signals generated during the translocation of the polymer through the nanopore channel to determine normalised measurement signal values; and generating a polymer sequence for each polymer of the series from the corresponding normalised measurement signal values for each polymer.

### RELATED APPLICATIONS

This application is a national stage filing under 35 U.S.C. § 371 of international application number PCT/GB2024/050173, filed Jan. 23, 2024, which claims the benefit of United Kingdom application number GB 2301095.2, filed Jan. 25, 2023, each of which is herein incorporated by reference in its entirety.

### TECHNICAL FIELD

The present invention relates to a method of calibrating a nanopore device. More particularly the invention relates to a method of calibrating a nanopore array device. Most particularly the invention relates to a method of calibrating a nanopore array device used for sensing molecular entities of an analyte.

### BACKGROUND

The use of nanopores to sense interactions with molecular entities, for example polynucleotides is a powerful technique that has been subject to much recent development. Nanopore devices have been developed that comprise an array of nanopore sensing elements, thereby increasing data collection by allowing plural nanopores to sense interactions in parallel, typically from the same sample.

### SUMMARY OF THE INVENTION

Nanopore devices may typically employ an electrical signal across a nanopore channel to generate a measurement signal that is interpreted to sense and/or characterise molecular entities as they interact with the nanopore. Typically an electrical signal is applied as a potential difference or current across the array of nanopore channels that will provide a meaningful measurement signal to be interpreted. The measurement can include, for example, one of ionic current flow, electrical resistance, or voltage.

Typically, the electrical signal for the array device is applied to the system at a predetermined value or values. Changes in the measurement signal over time can then be interpreted to determine the molecular entity present in the analyte. However, in an array device, variation across the array in extrinsic factors (i.e. standard conditions) or intrinsic factors (i.e. the “health” of the nanopore channel or membrane) can result in the variation of the measurement signals across the array and taint or skew the measurement signal from the nanopore channel.

In a first embodiment the present invention relates to a method of generating a polymer sequence using a nanopore sequencing device, the nanopore array device comprising a nanopore channel, the nanopore channel formed in a membrane separating two ionic solutions, the nanopore channel connecting the ionic solutions, the method comprising:
translocating a series of polymers through the nanopore channel; generating measurement signals during the translocation of each polymer through the nanopore channel; analysing a set of measurement signals from each polymer taken during the translocation of said polymer through the nanopore channel to determine a scaling factor for each polymer; applying the scaling factor to the measurement signals generated during the translocation of the polymer through the nanopore channel to determine normalised measurement signal values; and generating a polymer sequence for each polymer of the series from the corresponding normalised measurement signal values for each polymer.

By determining a scaling factor for each polymer that translocates through the pore a more accurate signal estimation for each polymer can be achieved. The scaling factor is determined from a set of measurements that arise from a region(s) of the polymer to ensure, as far as possible, a consistent signal level, substantially or wholly independent of the channel, pore, flowcell, or other experimental or standard conditions. In turn, the use of such a scaling factor improves the quality and robustness of the normalised signal for its use in generating a polymer sequence from, for example a basecaller or other computational sequence determining technique.

In addition, an improved read scaling, based on carefully chosen quantiles of the full-read distribution has been found to reduce genomic bias in polymers with low entropy (i.e. low variation). Furthermore, read-to-read noise and drifts from standard conditions between reads can be reduced using the method of the present invention when combined with scaling factors over measurement signals of multiple polymers. This is non-trivial since there are many variables that effect each channel such as temperature variations, electrolyte mediator concentration at the electrodes, the health of the nanopore and the membrane etc. Thus, it is important to compute a rolling statistic (i.e. a scaling factor determined for each polymer of the series) rather than global statistic (i.e. a scaling factor applied for the whole series of polymer) since the scaling properties of the nanopore channel can vary over longer timescales.

The set of measurement signals from each polymer taken during the translocation of said polymer through the nanopore channel to determine a scaling factor for each polymer can be the measurements of an adaptor region or portion thereof for each polymer. Adapters for use in nanopore sequencing of, for example, polynucleotides may comprise at least one single stranded polynucleotide or non-polynucleotide region. For example, Y-adapters for use in nanopore sequencing are known in the art, such as a disclosed in WO22243691 and WO21255476, herein disclosed by reference in their entirety.

In this connection, the scaling factor determined from the adapter region for each polymer in the series of polymers also provides an estimate of changes within the system. This is more accurate than relying on a measurement signal taken from a localised non-adapter region from the polymer since the sequence/content of the adaptor region is known for each polymer of the series, and is of known length. Thus, the use of an adapter region to determine a scaling factor ensures that a known region of polymer is used. This in turn means that the user is not reliant on an estimation of a sequence within in the read, or the polymer itself to determine the scaling factors since both of these measurements require the user to know what part of the polymer is to be used for scaling, and/or can rely on a post-facto analysis about what has been sequenced prior to applying scaling. The adapter may be the same for all polymers in the series of polymers. This means that in a perfect system there should be no variation in the scaling factor determined from the measurements generated by the nanopore channel. This can be useful in assessing extrinsic/intrinsic changes to the nanopore channel during use.

In addition, the adaptor region is often the first part of the polymer that is sequenced, and is often of a known length. And so the determination of a scaling factor can be accurately determined from analysis of the known adaptor. This scaling based on an adapter region can be further improved by determining the median signal and excluding any signal measurements that are above/below certain quantiles (typical 10 (i.e. lower 5^{th} and upper 95^{th} quantiles), even more typically 20 (i.e. lower 10^{th} and upper 90^{th} quantiles)). This ensures that extraneous or erroneous data points are excluded from the scaling applied to the measurement signal.

At least one polymer in the series of polymers may be shorter in length than the adaptor region of said polymer. In this instance the polymer could be a short strand or fragment of a larger polymer. This can mean that the fragmented portions are parts of a fragmented target polymer which can be reconstructed to determine the overall target polymer sequence. The method of the present invention is particularly suited for generating a scaling factor for polymers of this length since the adaptor region provides a consistent and reliable set of measurements that can be used for scaling much shorter sets of measurements. In addition, the polymer to be analysed may not be long enough to provide enough measurements for an accurate scaling factor, and that, for example, signal noise might carry more weight in the determining of the scaling factor and thus adversely affect the accuracy of the scaling factor. The reconstructed sequence can be said to be constructed from more accurate sequencing estimates based on the scaling factor applied to each fragment.

The scaling factors from the series of polymers may be analysed to determine the condition of the nanopore channel nanopore device. As mentioned above, the scaling properties of the nanopore channel can vary over longer timescales. By analysing the changes in the scaling factor from each of the polymers, especially if a consistent adaptor region is used for each polymer, the user can be notified if there are abnormal or unexpected drifts or changes which might indicate that the nanopore channel is not functioning as expected due to, for example, temperature changes, damage to the nanopore or membrane, and electrolyte mediator depletion in system.

The nanopore sequencing device may comprise an array of nanopore channels. In this case, the change in scaling factors per channel can be monitored by the user. In this scenario, the nanopore sequencing device may select a nanopore channel from the array based on the scaling factor or change in scaling factors associated with said nanopore channel. The method of the present invention can be used so the device can be configured to select functioning nanopore channels from the array of nanopore channels based on reviewing scaling factors or changes in scaling factors from individual channels.

The nanopore in the nanopore channel may be a biological pore supported on membrane. The nanopore may be a solid-state pore or a biological pore supported on a solid-state membrane. The nanopore device may comprise a sensor electrode and the measurements comprise electrical measurements. Furthermore, the nanopore device may comprise a sensor electrode, and the measurements taken by the sensor are indicative of ion flow through the nanopore.

The polymer may comprise a series of polymer units to be identified by the nanopore device. The polymer is a polynucleotide, and the polymer units are nucleotides. In a particular embodiment the polymer or polynucleotide is genomic DNA. The use of the present invention is particularly beneficial for polymers such as genomic DNA and RNA wherein there can be said to be low entropy in the molecular moieties to be sensed. In other words, the improved scaling based on a region of the polymer translocating through the nanopore channel provides a better fidelity or resolution to samples with highly repetitive patterns of particular molecular moieties For instance, genomic DNA can be considered to have regions with repeats of G and C bases which can be miscounted or misinterpreted by the methods of the prior art.

In examples a median of the measurement signal value can be determined and used in the determination of the normalised measurement signal values. This enables an improvement by determining the median signal and excluding any signal measurements that are above/below certain quantiles (typical 10 (i.e. lower 5^{th} and upper 95^{th} quantiles). This ensures that extraneous or erroneous data points are excluded from the scaling applied to the measurement signal.

### DESCRIPTION OF THE FIGURES

To allow better understanding, embodiments of the present invention will now be described by way of non-limitative example with reference to the accompanying drawings, in which:

FIG. 1 is diagram of a nanopore array device;

FIG. 2 is a schematic cross-sectional view of part of a nanopore array device;

FIG. 3 shows three example traces from a nanopore channel as a polymer translocates through the nanopore channel with the x axis indicating time, and the y axis indicating ionic current; and

FIG. 4 a plot showing polymers of varying GC content plotted against the quantile of data included along the x axis and the median ionic current value plotted along the y axis.

### DETAILED DESCRIPTION

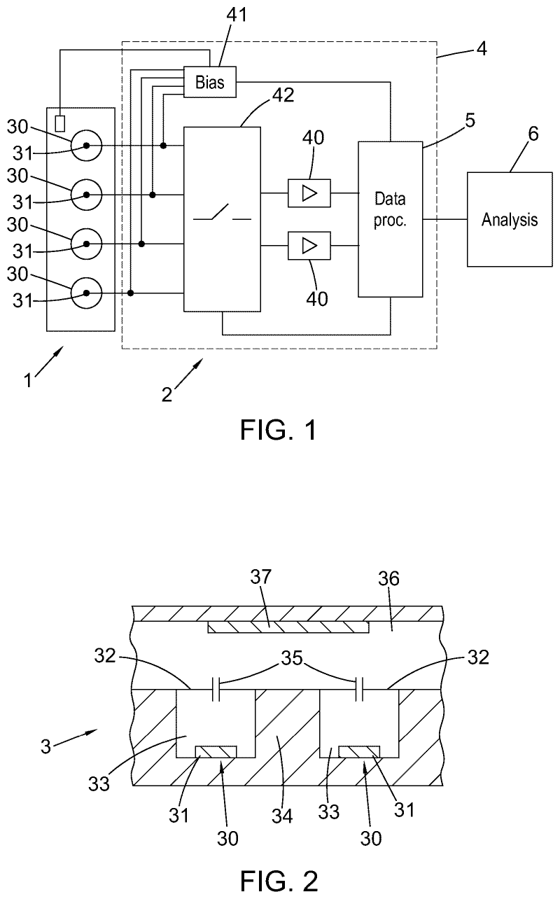

A nanopore array device 1 for sensing interactions of a molecular entities is shown in FIG. 1. The nanopore array device 1 comprises a sensing apparatus 2 comprising a sensor device 3 and a detection circuit 4 that is connected to the sensor device 3.

The sensor device 3 comprises an array of sensing elements 30 that each support respective nanopores that are capable of an interaction with a molecular entity. The sensing elements 30 comprise respective electrodes 31. In use, each sensing elements 30 outputs an electrical measurement at its electrode 31 that is dependent on an interaction of a molecular entity with the nanopore. The sensor device 3 is illustrated schematically in FIG. 1 but may have a variety of configurations, some non-limitative examples being as follows.

In one example, the sensor device 3 may have the form shown in FIG. 2. Herein, the sensor device 2 comprises an array of sensing elements 30 which each comprise a membrane 32 supported across a well 33 in a substrate 34 with a nanopore 35 inserted in the membrane 32. The membrane 31 may be made of amphiphilic molecules such as lipid as discussed further below. Each membrane 32 seals the respective well 33 from a sample chamber 36 which extends across the array of sensing elements 30 and is in fluid communication with each nanopore 35. Each well 33 has a sensor electrode 32 arranged therein. A common electrode 37 is provided in the sample chamber 36 for providing a common reference signal (typically a potential or voltage) to each sensor element 30. In use, the sample chamber 36 receives a sample containing molecular entities which interact with the nanopores 35 of the sensing elements 30.

Two sensing elements 30 are shown in FIG. 2 for clarity, but in general any number of sensing elements 30 may be provided. Typically, a large number of sensing elements 30 may be provided to optimise the data collection rate, for example 256, 1024, 4096 or more sensing elements 30.

The sensor device 3 may have a detailed construction as disclosed in WO 2009/077734 or WO 2014/064443 which are herein incorporated by reference in their entireties.

The nanopore and associated elements of the sensing elements 30 may be as follows, without limitation to the example shown in FIG. 2.

The nanopore is a pore, typically having a size of the order of nanometres. In embodiments where the molecular entities are polymers that interact with the nanopore while translocating therethrough in which case the nanopore is of a suitable size to allow the passage of polymers therethrough.

The nanopore may be a protein pore or a solid-state pore. The dimensions of the pore may be such that only one polymer may translocate the pore at a time.

Where the nanopore is a protein pore, it may have the following properties.

The nanopore may be a transmembrane protein pore. Transmembrane protein pores for use in accordance with the invention include, but are not limited to, β-toxins, such as α-hemolysin, anthrax toxin and leukocidins, and outer membrane proteins/porins of bacteria, such as Mycobacterium smegmatis porin (Msp), for example MspA, lysenin, outer membrane porin F (OmpF), outer membrane porin G (OmpG), outer membrane phospholipase A and Neisseria autotransporter lipoprotein (NalP). α-helix bundle pores comprise a barrel or channel that is formed from a-helices. Suitable α-helix bundle pores include, but are not limited to, inner membrane proteins and α outer membrane proteins, such as WZA and ClyA toxin. The transmembrane pore may be derived from lysenin. The pore may be derived from CsgG, such as disclosed in WO-2016/034591 which is herein incorporated by reference in its entirety. The pore may be a DNA origami pore.

The protein pore may be a naturally occurring pore or may be a mutant pore. The pore may be fully synthetic.

Where the nanopore is a protein pore, it may be inserted into a membrane that is supported in the sensor element 30. Such a membrane may be an amphiphilic layer, for example a lipid bilayer. An amphiphilic layer is a layer formed from amphiphilic molecules, such as phospholipids, which have both hydrophilic and lipophilic properties. The amphiphilic layer may be a monolayer or a bilayer. The amphiphilic layer may be a co-block polymer such as disclosed in WO 2014/064444. Alternatively, a protein pore may be inserted into an aperture provided in a solid-state layer, for example as disclosed in WO 2012/005857.

The nanopore may comprise an aperture formed in a solid-state layer, which may be referred to as a solid-state pore. The aperture may be a well, gap, channel, trench or slit provided in the solid-state layer along or into which analyte may pass. Solid-state layers can be formed from both organic and inorganic materials including, but not limited to, microelectronic materials, insulating materials such as Si3N4, A12O3, and SiO, organic and inorganic polymers such as polyamide, plastics such as Teflon® or elastomers such as two-component addition-cure silicone rubber, and glasses. The solid-state layer may be formed from graphene.

Molecular entities interact with the nanopores in the sensing elements 30 causing output an electrical signal at the electrode 31 that is dependent on that interaction.

In one type of sensor device 3, the electrical signal may be the ion current flowing through the nanopore. Similarly, electrical properties other than ion current may be measured. Some examples of alternative types of property include without limitation: ionic current, impedance, a tunnelling property, for example tunnelling current (for example as disclosed in Ivanov AP et al., Nano Lett. 2011 Jan. 12; 11(1):279-85 which is herein incorporated by reference in its entirety), and a FET (field effect transistor) voltage (for example as disclosed in WO2005/124888 which is herein incorporated by reference in its entirety). One or more optical properties may be used, optionally combined with electrical properties (Soni GV et al., Rev Sci Instrum. 2010 January;81(1):014301 which is herein incorporated by reference in its entirety). The property may be a transmembrane current, such as ion current flow through a nanopore. The ion current may typically be the DC ion current, although in principle an alternative is to use the AC current flow (i.e. the magnitude of the AC current flowing under application of an AC voltage).

The interaction may occur during translocation of the molecular entities with respect to the nanopore, for example through the nanopore.

The electrical signal provides as series of measurements of a property that is associated with an interaction between the molecular entity and the nanopore. Such an interaction may occur at a constricted region of the nanopore. For example in the case that the molecular entity is a polymer comprising a series of polymer units which translocate with respect to the nanopore, the measurements may be of a property that depends on the successive polymer units translocating with respect to the pore.

Ionic solutions may be provided on either side of the nanopore. A sample containing the molecular entities of interest that are polymers may be added to one side of the nanopore, for example in the sample chamber 36 in the sensor device of FIG. 2. membrane and allowed to translocate with respect to the nanopore, for example under a potential difference or chemical gradient. The electrical signal may be derived during the translocation of the polymer with respect to the pore, for example taken during translocation of the polymer through the nanopore. The polymer may partially translocate with respect to the nanopore.

In order to allow measurements to be taken as a polymer translocates through a nanopore, the rate of translocation can be controlled by a binding moiety that binds to the polymer. Typically the binding moiety can move a polymer through the nanopore with or against an applied field. The binding moiety can be a molecular motor using for example, in the case where the binding moiety is an enzyme, enzymatic activity, or as a molecular brake. Where the polymer is a polynucleotide there are a number of methods proposed for controlling the rate of translocation including use of polynucleotide binding enzymes. Suitable enzymes for controlling the rate of translocation of polynucleotides include, but are not limited to, polymerases, helicases, exonucleases, single stranded and double stranded binding proteins, and topoisomerases, such as gyrases. For other polymer types, binding moieties that interact with that polymer type can be used. The binding moiety may be any disclosed in WO-2010/086603, WO-2012/107778, and Lieberman KR et al, J Am Chem Soc. 2010;132(50):17961-72), and for voltage gated schemes (Luan B et al., Phys Rev Lett. 2010;104(23):238103) which are all herein incorporated by reference in their entireties.

The binding moiety can be used in a number of ways to control the polymer motion. The binding moiety can move the polymer through the nanopore with or against the applied field. The binding moiety can be used as a molecular motor using for example, in the case where the binding moiety is an enzyme, enzymatic activity, or as a molecular brake. The translocation of the polymer may be controlled by a molecular ratchet that controls the movement of the polymer through the pore. The molecular ratchet may be a polymer binding protein.

The polynucleotide handling enzyme may be for example one of the types of polynucleotide handling enzyme described in WO 2015/140535 or WO-2010/086603.

Translocation of the polymer through the nanopore may occur, either cis to trans or trans to cis, either with or against an applied potential. The translocation may occur under an applied potential which may control the translocation.

Exonucleases that act progressively or processively on double stranded DNA can be used on the cis side of the pore to feed the remaining single strand through under an applied potential or the trans side under a reverse potential. Likewise, a helicase that unwinds the double stranded DNA can also be used in a similar manner. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential. Alternatively, the single strand DNA dependent polymerases can act as a molecular brake slowing down the movement of a polynucleotide through the pore. Any moieties, techniques or enzymes described in WO-2012/107778 or WO-2012/033524 which are both herein incorporated by reference in their entireties could be used to control polymer motion.

The sensing elements 30 and/or the molecular entities may be adapted to capture molecular entities within a vicinity of the respective nanopores. For example sensing elements 30 may further comprise capture moieties arranged to capture molecular entities within a vicinity of the respective nanopores. The capture moieties may be any of the binding moieties or exonucleases described above with also have the purpose of controlling the translocation or may be separately provided.

The capture moieties may be attached to the nanopores of the sensing elements. At least one capture moiety may be attached to the nanopore of each sensor element.

The capture moiety may be a tag or tether which binds to the molecular entities. In that case the molecular entity may be adapted to achieve that binding.

Such a tag or tether may be attached to the nanopore, for example as disclosed in WO 2018/100370 which is herein incorporated by reference in its entirety, and as further described herein below.

Alternatively in a case the nanopore is inserted in a membrane, such a tag or tether may be attached to the membrane, for example as disclosed in WO 2012/164270 which is herein incorporated by reference in its entirety.

The methods described herein may comprise the use of adapters which adapt the polymers for the purpose of optimising their translocation through the nanopore. The adaptor is typically ligated onto one or both ends of the polymer may comprise one or more spacers for stalling a motor protein loaded onto the adapter. By way of example, polynucleotide adapters suitable for use in nanopore sequencing of polynucleotides are known in the art. Adapters for use in nanopore sequencing of polynucleotides may comprise at least one single stranded polynucleotide or non-polynucleotide region. For example, Y-adapters for use in nanopore sequencing are known in the art, such as a disclosed in WO22243691 and WO21255476, herein disclosed by reference in their entirety. A Y adapter typically comprises (a) a double stranded region and (b) a single stranded region or a region that is not complementary at the other end. A Y adapter may be described as having an overhang if it comprises a single stranded region. The presence of a non-complementary region in the Y adapter gives the adapter its Y shape since the two strands typically do not hybridise to each other unlike the double stranded portion. The Y adapter may comprise one or more anchors.

The Y adapter preferably comprises a leader sequence which preferentially threads into the pore. The leader sequence typically comprises a polymer. The polymer is preferably negatively charged. The polymer is preferably a polynucleotide, such as DNA or RNA, a modified polynucleotide (such as abasic DNA), PNA, LNA, polyethylene glycol (PEG) or a polypeptide. The leader preferably comprises a polynucleotide and more preferably comprises a single stranded polynucleotide. The adapter may be ligated to a DNA molecule using any method known in the art. Each polymer will typically comprise the same adapter molecule and thus measurement of the adapter region or portion thereof may advantageously be used to provide a reliable scaling factor.

A polynucleotide adapter may comprise a membrane anchor or a transmembrane pore anchor attached to the adapter. For example, a membrane anchor or transmembrane pore anchor may promote localisation of the adapter and coupled polynucleotide within a vicinity of the nanopore. The anchor may be a polypeptide anchor and/or a hydrophobic anchor that can be inserted into the membrane. In one embodiment, the hydrophobic anchor is a lipid, fatty acid, sterol, carbon nanotube, polypeptide, protein or amino acid, for example cholesterol, palmitate or tocopherol.

The anchor may comprise a linker, or 2, 3, 4 or more linkers. Preferred linkers include, but are not limited to, polymers, such as polynucleotides, polyethylene glycols (PEGs), polysaccharides and polypeptides. These linkers may be linear, branched or circular. Suitable linkers are described in WO 2010/086602. Examples of suitable anchors and methods of attaching anchors to adapters are disclosed in WO 2012/164270 and WO 2015/150786 which are both herein incorporated by reference in their entireties.

Examples of tags and tethers which are attached to the nanopore are as follows.

Nanopores for use in the methods described herein may be modified to comprise one or more binding sites for binding to one or more analytes (e.g. molecular entities) and thereby acting as a capture moiety. In some embodiments, the nanopores may be modified to comprise one or more binding sites for binding to an adaptor attached to the analytes. For example, in some embodiments, the nanopores may bind to a leader sequence of the adaptor attached to the analytes. In some embodiments, the nanopores may bind to a single stranded sequence in the adaptor attached to the analytes.

In some embodiments, the nanopores are modified to comprise one or more tags or tethers, each tag or tether comprising a binding site for the analyte. In some embodiments, the nanopores are modified to comprise one tag or tether per nanopore, each tag or tether comprising a binding site for the analyte.

In some embodiments, the tag or tether may comprise or be an oligonucleotide.

Other examples of a tag or tether include, but are not limited to His tags, biotin or streptavidin, antibodies that bind to analytes, aptamers that bind to analytes, analyte binding domains such as DNA binding domains (including, e.g., peptide zippers such as leucine zippers, single-stranded DNA binding proteins (SSB)), and any combinations thereof.

The tag or tether may be attached to the external surface of the nanopore, e.g., on the cis side of a membrane, using any methods known in the art. For example, one or more tags or tethers can be attached to the nanopore via one or more cysteines (cysteine linkage), one or more primary amines such as lysines, one or more non-natural amino acids, one or more histidines (His tags), one or more biotin or streptavidin, one or more antibody-based tags, one or more enzyme modification of an epitope (including, e.g., acetyl transferase), and any combinations thereof. Suitable methods for carrying out such modifications are well-known in the art. Suitable non-natural amino acids include, but are not limited to, 4-azido-L-phenylalanine (Faz) and any one of the amino acids numbered 1-71 in FIG. 1 of Liu C. C. and Schultz P. G., Annu. Rev. Biochem., 2010, 79, 413-444 which is herein incorporated by reference in its entirety.

In some embodiments where one or more tags or tethers are attached to the nanopore via cysteine linkage(s), the one or more cysteines can be introduced to one or more monomers that form the nanopore by substitution.

The transmembrane pore may be modified to enhance capture of polynucleotides. For example, the pore may be modified to increase the positive charges within the entrance to the pore and/or within the barrel of the pore. Such modifications are known in the art. For example, WO 2010/055307 discloses mutations in a-hemolysin that increase positive charge within the barrel of the pore.

Modified MspA, lysenin and CsgG pores comprising mutations that enhance polynucleotide capture are disclosed in WO 2012/107778, WO 2013/153359 and WO 2016/034591, respectively which are all herein incorporated by reference in their entireties. Any of the modified pores disclosed in these publications may be used herein.

The arrangement of the detection circuit 4 will now be discussed. The detection circuit 4 is connected to the electrodes 31 of each sensor element 30 and has the primary function of process the electrical signals output therefrom. The detection circuit 4 also has the function of controlling the application of bias signals to each sensor element 30.

The detection circuit 4 includes plural detection channels 40. Each detection channel 40 receives an electrical signal from a single sensor electrode 31and is arranged to amplify that electrical signal. The detection channel 40 is therefore designed to amplify very small currents with sufficient resolution to detect the characteristic changes caused by the interaction of interest. The detection channel 40 is also designed with a sufficiently high bandwidth to provide the time resolution needed to detect each such interaction. These constraints require sensitive and therefore expensive components. Each detection channel 40 may be similar to standard single channel recording equipment as describe in Stoddart D et al., Proc Natl Acad Sci, 12;106(19):7702-7, Lieberman KR et al, J Am Chem Soc. 2010;132(50):17961-72, and WO-2000/28312. Alternatively, each detection channel 40 may be arranged as described in detail in WO 2010/122293, WO 2011/067559 or WO 2016/181118.

The analyte of interest to be detected by the nanopore may be a polynucleotide such as DNA or RNA. The nucleotides may be naturally or non-naturally occurring. The nucleotides may be modified. In particular the polynucleotide may be genomic DNA or genomic RNA and the method of the invention may be used to more accurately determine GC bias in genomes. The genome may be derived from any source and may be eukaryotic or prokaryotic. The genome may be bacterial, viral, plant, animal, algal, protozoal or archaeal. The analyte may be a polypeptide or a polysaccharide.

The number of sensing elements 30 in the array is greater than the number of detection channels 40 and the nanopore array device is operable to take measurements of a polymer from sensing elements 30 selected in a multiplexed manner, in particular an electrically multiplexed manner. This is achieved by providing a switch arrangement 42 between the sensor electrodes 31 of the sensing elements 30 and the detection channels 40. For clarity, FIG. 1 shows a simplified example with four sensing elements 30 and two detection channels 40, but the number of sensor cells 30 and detection channels 40 is typically much greater. For example, for some applications, the sensor device 2 might comprise a total of 4096 sensing elements 30 and 1024 detection channels 40.

The switch arrangement 42 may be arranged as described in detail in WO 2010/122293. For example, the switch arrangement 42 may comprise plural 1-to-N multiplexers each connected from a detection channel 40 to a group of N sensing elements 30 and may include appropriate hardware such as a latch to select the state of the switching.

By switching of the switch arrangement 42, the nanopore array device 1 may be operated to amplify electrical signals from sensing elements 30 selected in an electrically multiplexed manner. The detection circuit 4 includes a data processor 5 which receives the output signals from the detection channels 40. The data processor 5 acts as a controller that controls the switch arrangement 42 to connect detection channels 40 to respective sensing elements 30 as described further below.

In addition, the detection circuit 4 includes a bias control circuit 41 to perform the function of controlling the application of bias signals to each sensor element 30. The bias control circuit 41 is connected to the common electrode 37 and to the sensor electrodes 31 of each sensor device 30. The bias signals are selected to bias the sensor electrodes 31 with respect to common electrode 37 to control translocation of the molecular entities with respect to the nanopores. In general, it would be possible for a bias signal supplied to a given sensor element 30 to be a drive bias signal that causes translocation to occur at the sensor element 30 or an inhibition bias signal that inhibits translocation to occur at the sensor element 30.

The bias control circuit 41 is controlled by the data processor 5. The data processor has a mode of operation for the bias control circuit 41. Namely, three independent test bias signals are supplied to all the sensing elements 30, thereby causing ionic current flow with respect to the nanopores of each sensing elements 30. The corresponding current flow for each test signal is recorded in the data processor 5 as an amplified electrical signal.

The data processor 5 is arranged as follows. The data processor 5 is connected to the output of the detection channels 40 and is supplied with the amplified electrical signals therefrom. The data processor 5 stores and analyses the amplified electrical signals from the test bias signals to create a calibrated signal. The data processor 5 also controls the other elements of the detection circuit, including control of the bias voltage circuit 41 as described above and control of the switch arrangement 42 as described below. The data processor 5 forms part of the detection circuit 2 and may be provided in a common package therewith, possibly on a common circuit board. The data processor 5 may be implemented in any suitable form, for example as a processor running an appropriate computer program or as an ASIC (application specific integrated circuit).

The data processor 5 of the nanopore array device 1 is connected to an analysis system 6. The data processor 5 also supplies the amplified output signals to the analysis system 6. The analysis system 6 performs further analysis of the amplified electrical signal which is a raw signal representing measurements of the property measured at the nanopore. Such an analysis system 6 may for example estimate the identity of the molecular entity in its entirety or in the case that the molecular entity is a polymer may estimate the identity of the polymer units thereof. Thus, the analysis system may be configured as a computer apparatus running an appropriate program. Such a computer apparatus may be connected to the data processor 5 of the nanopore array device 1 directly or via a network, for example within a cloud-based system.

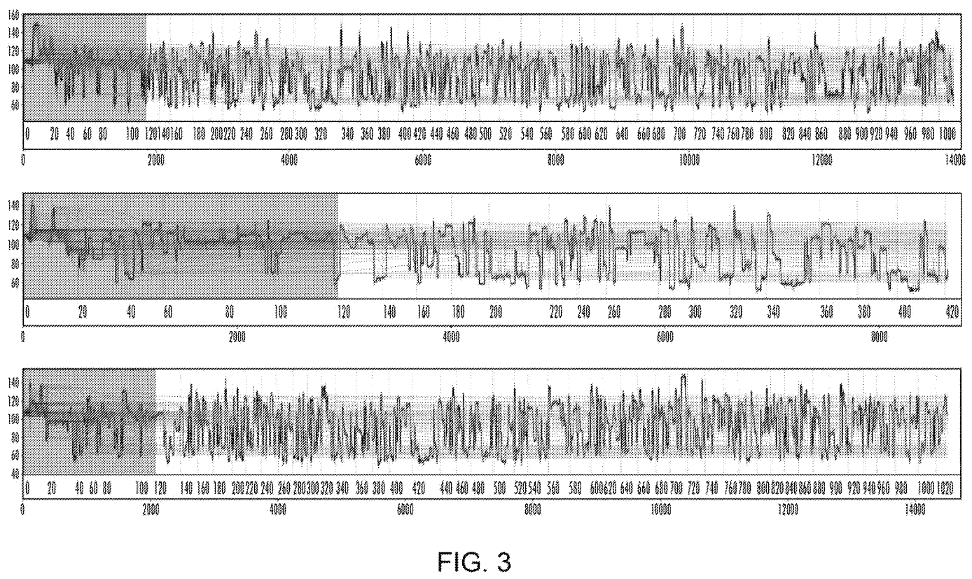

FIG. 3 provides three example traces which show the measurement signals from three different polymers that are translocating through a nanopore channel. The polymers are different strands of DNA or DNA fragments of varying sequences and lengths.

The area shaded from time 0 onwards is representative of a region where a set of measurements are taken for normalising the signal across the whole of the measurements generated from the polymer. In these particular examples the region of interest for scaling purposes is the adaptor region of the polymer. As shown in the example traces, the adapter region is not always the same length or sequence.

The trace varying in amplitude along the x-axis is the general trace of ionic current through the nanopore channel as the polymer translocates through the nanopore channel. The change in ionic current is indicative of the molecular moiety that is within the nanopore channel and this change in measurement signal can be interpreted using computational methods, such as a base caller.

In the present examples, these traces of measurement signal are normalised against a scaling factor which is generated when comparing the trace in the shaded region against either values which are expected for that region, or previous readings of that same region from another polymer in a series of polymers that are being analysed. These normalised measurement signals are then much easier to interpret by, for example, a base caller or another computational program that can interpret complex signals.

In addition, there are running parallel with the x-axis a set of lines showing the quantiles of the signal measurements that are calculated from the median value of the signal generated as the polymer translocates through the nanopore.

These quantiles can be used to exclude extreme values measured in the region of interest for scaling, and thus improve the general accuracy of the scaling factor to be applied.

An issue with using a whole-strand based scaling approach is that the computed scaling factor would depend on the genomic content of the read and not just on scaling properties of the nanopore channel. When using a scaling factor generated across data taken from whole strands translocating through a nanopore channel, the aim would be to generate a more precise scaling parameters which a program like a base call model would learn to rely upon during training.

This in turn may exacerbate the issue of bias (i.e. resolving the amount of G or C homogeneity within a strand region, this is seen particularly in genomic polymers such as DNA and RNA).

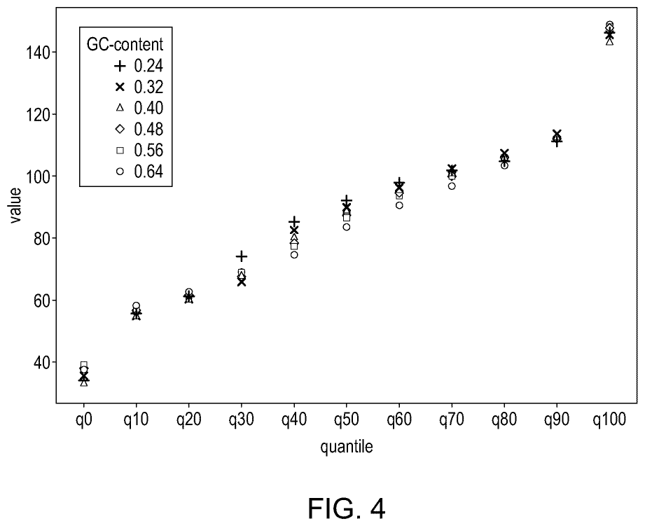

To illustrate the issue, FIG. 4 provides a plot of median values from the signal quantiles for different genomes. To highlight the main effect, GC content has been computed from the main genomes within the dataset. The plot shows signal quantiles for different genomes, shaded according to GC-content. The median (q50) and nearby quantiles have a wide spread of values which depends strongly on GC content of the genome. On the other hand 20th and 90th quantiles are tightly clustered across genomes. This approach excludes the extremities of the measurement signals from things such as noise etc when generating the scaling factor and normalising the data. This improves the general accuracy and fidelity of the data as it is fed into a base caller, for example, to generate a polymer sequence from the normalised measurement signal.

1. A method of generating a polymer sequence using a nanopore sequencing
device,
the nanopore array device comprising a nanopore channel, the nanopore channel formed in a membrane separating two ionic solutions, the nanopore channel connecting the ionic solutions,
the method comprising:
translocating a series of polymers through the nanopore channel;
generating measurement signals during the translocation of each polymer through the nanopore channel;
analysing a set of measurement signals from each polymer taken during the translocation of said polymer through the nanopore channel to determine a scaling factor for each polymer;
applying the scaling factor to the measurement signals generated during the translocation of the polymer through the nanopore channel to determine normalised measurement signal values; and
generating a polymer sequence for each polymer of the series from the corresponding normalised measurement signal values for each polymer.

2. The method according to claim 1, wherein the set of measurement signals from each polymer taken during the translocation of said polymer through the nanopore channel to determine a scaling factor for each polymer is the measurements of an adaptor region of each polymer.

3. The method according to claim 2, wherein at least one polymer in the series of polymers is shorter in length than the adaptor region of said polymer.

4. The method according to any one of the preceding claims, wherein the scaling factors are analysed over the series of polymers to determine the condition of the nanopore channel nanopore device.

5. The method according to any one of the preceding claims, wherein the nanopore sequencing device comprises an array of nanopore channels, and wherein measurement signals corresponding to the polymers are generated during translocation through the respective nanopore channels.

6. The method according to claim 5, wherein the nanopore sequencing device selects a nanopore channel from the array based on the scaling factor or scaling factors associated with said nanopore channel.

7. A method according to any one of the preceding claims, wherein the nanopore is a biological pore.

8. A method according to any one of the preceding claims, wherein the polymer comprises a series of polymer units to be identified by the nanopore device.

9. A method according claim 8, wherein the polymer is a polynucleotide, and the polymer units are nucleotides.

10. A method according claim 8 or 9, wherein the polymer or polynucleotide is genomic DNA.

11. A method according to any one of the preceding claims, wherein a median of the measurement signal value is determined and used in the determination of the normalised measurement signal values.

12. A method according to any one of the preceding claims, wherein the nanopore device comprises a sensor electrode and the measurements comprise electrical measurements.

13. The method according to any one of the previous claims, wherein the nanopore device comprises a sensor electrode, and the measurements taken by the sensor are indicative of ion flow through the nanopore.
